(19) United States
(12) Patent Application Publication
(10) Pub. No.: US 2016/0354174 A1
(43) Pub. Date: Dec. 8, 2016

(54) WIRELESS STRAIN SENSING DEVICE

(71) Applicant: INNOVATIVE IN VIVO SENSING, LLC, Fort Collins, CO (US)

(72) Inventor: Hilmi Volkan DEMIR, Ankara (TR)

(21) Appl. No.: 15/173,607

(22) Filed: Jun. 3, 2016

Related U.S. Application Data
(60) Provisional application No. 62/172,026, filed on Jun. 5, 2015.

(51) Int. Cl. A61B 90/00; A61B 5/00; G01B 7/16

(52) U.S. Cl. CPC A61B 90/06; G01B 7/16; A61B 5/0031; A61B 5/686; A61B 2090/064; A61B 2562/0261

(57) ABSTRACT

A wireless strain sensing device uses a plurality of wireless sensors to obtain individual local strain data on the spot (area) where the wireless sensor is disposed on a stress member using a specific frequency response that is scanned externally. The resonance frequency shifts accordance with the locally applied strain on the specific point of the stress member. The change in the resonance frequency in response to the local strain can then be allocated within a limited spectral range for each of the wireless sensors. This permits a plurality of wireless sensors, each having a different initial resonance frequency, to be read concurrently and distinguished.

This application claims the priority benefit of U.S. Provisional Patent Application Ser. No. 62/172,026 filed 5 Jun. 2015. The disclosure of thereof, in its entirety, is incorporated herein by reference.

### BACKGROUND

An implant device is commonly subject to mechanical loading in a complex way inside the body. Consequently, the strain buildup in the implant exhibits a complex distribution, which makes it very challenging and typically impossible to correlate the actual loading situation to a single strain reading from the implant.

U.S. Pat. No. 9,326,728 discloses a wireless resonator sensor circuitry, composed of a dielectric material and a conductive coil that functions as a strain gauge. The detailed description of the resonator sensor circuitry disclosed in U.S. Pat. No. 9,326,728 is herein incorporated by reference.

The acute hospital readmission rates for patients with tibial or femoral fractures exceed 15%, with greater than 50% of these attributable to non-unions. Currently available technology cannot predict the course of aberrant fracture during the critically-important early healing phase (<4 weeks post-operative). This represents a significant clinical detriment and impedes decision-making with regard to whether the administration of additional therapies (such as biologics) should be implemented.

There is a need for multiple local strain readings simultaneously or concurrently from multiple areas of the implant.

### SUMMARY

One aspect of the present invention relates to a wireless strain sensing device that enables wireless reading from multiple areas of a stress member. The strain sensing device includes a stress member and a plurality of local strain sensors, each allocated and dedicated for reading from a single target area of the stress member.

Specifically, the wireless strain sensing device includes a stress member and a plurality of wireless resonating strain sensors disposed at different areas on the stress member. Each of the wireless resonating stain sensors comprises a strain sensing circuit that functions as a strain gauge and a resonator and composed of a first dielectric layer composed of dielectric material disposed over the stress member, and a first conductive layer disposed over the first dielectric layer. Each of the wireless resonating strain sensors is configured to have a resonant frequency that is spectrally separated from each other to enable each of the resonant frequencies of the wireless resonating strain sensors to be distinguishable from each other even when the strain applied to the stress member causes shifting of the resonance frequency thereof.

The first conductive layer has a patterned layout and the stress member is made of a conductive material. Part of the first conductive layer can be in electrical contact with the stress member through the first dielectric layer.

Each of the wireless resonating strain sensors can further include a second dielectric layer disposed over the first conductive layer and a second conductive layer disposed the over the second dielectric layer. The second conductive layer has a patterned layout. The first conductive layer also can have a patterned layout.

The stress member is made of metal

The stress member can be a conductive implant hardware.

The stress member can be flexible and conductive.

The sensors can be directly patterned or printed on the target areas of the stress member, avoiding the need for a substrate. That is, the stress member itself can act as a substrate. The sensors can be an inductively-powered telemetric wireless sensor (i.e., no battery source). That is, the sensors can be fully passive and integrated into the stress member. The resulting system thus does not contain any battery or any other standalone component internal to the area where the sensing device is embedded, attached, or buried, except for the stress member itself. Multiple individual areas can be read directly from the stress member concurrently in real time wirelessly from the stress member.

The ability to concurrently read multiple areas of the stress member provides individual local strain readings from each of the sensors simultaneously. This information can be used to acquire and understand actual complex strain buildup that the stress member undergoes.

### BRIEF DESCRIPTION OF THE DRAWINGS

FIG. 1 schematically illustrates a plurality of wireless sensors f1-fn, each having individual operating frequencies that accommodate a Δf frequency shift margin, on an implant hardware.

FIGS. 2A-2B schematically illustrate wireless sensors f1-fn of FIG. 1 with different antenna schemes.

FIGS. 3A-3B schematically illustrate one embodiment of the present device having f1-fn number of sensors using a single metal layer structure (only one sensor shown).

FIGS. 4A-4B schematically illustrate another embodiment similar to FIGS. 3A-3B, except that each sensor uses a double metal layer structure with one patterned metal layer and one unpatterned metal layer.

FIGS. 5A-5B schematically illustrate another embodiment similar to FIGS. 4A-4B, except that each sensor uses a double metal layer structure with two patterned metal layers.

### DETAILED DESCRIPTION

The present wireless strain sensing device telemetrically reports strain data from multiple points of the stress member SM. For instance, when it is used as a fracture fixation implant device, the data can be used to predict normal and aberrant fracture healing cascades during the acute fracture healing period in a large animal model because temporal implant strain correlates with callus healing.

The stress member can be a flexible substrate so that wireless sensors can be disposed at multiple locations on the same substrate, even with a curved surface, thereby drastically expanding this technology's potential utility and application to help answer important basic science questions. An example of a flexible substrate includes a vacuum tape, such as a KAPTON tape, which is made of polyamide or other flexible plastic material. Flexible tapes typically are composed of some type of organic materials that are robust and self-supporting. Another example includes an ultrathin flexible glass substrate. For example, in relation to an implant device, it is generally accepted that the mechanical environment imposed on the fracture callus be predominately controlled by the implant stiffness and a key parameter with respect to the ultimate healing outcome. But due to the large spectrum of fracture types/severities and internal fixation implant designs, there exists considerable controversy as to if there exists an optimal implant stiffness that results in successful clinical outcomes. The present flexible substrate technology platform can be used to utilize a multi-sensor configuration on a single implant hardware to measure the temporal and spatial implant strain profiles during bony healing, providing a measure of the unique in vivo variations with respect to the transient mechanical environment (i.e., implant strain profile) that are associated with specific implant designs and fracture type/severity combinations. If used as an implant, the strain sensing device would be biocompatible, which can be microelectromechanical rigid substrate sensors (bioMEMS) or flexible substrate sensors (fs-bioMEMS). The sensors integrated with the stress member, which can be made of metal, such as titanium, stainless steel, etc., that can take stress.

In operation, individual local strain data can be obtained on the spot (area) where the wireless sensor is disposed on the stress member using a specific frequency response that is scanned externally. Here the designed unit's resonance frequency shifts by the locally applied strain on the specific point of the stress member. The change in the resonance frequency in response to the local strain can then be allocated within a limited spectral range for each of the sensors. This permits a plurality of wireless sensors, each having a different initial resonance frequency, to be concurrently read and distinguished therebetween.

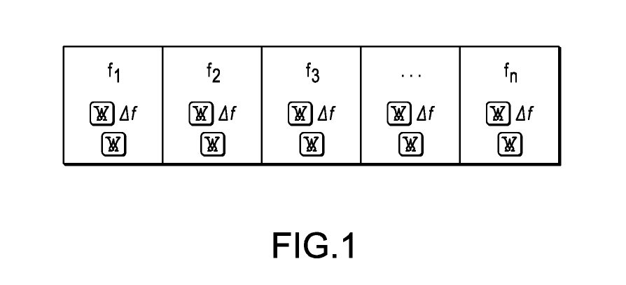

To provide multiple simultaneous readings of the local strain data from different parts of the same stress member SM, each wireless sensors need to be distinguished from each other. The wireless sensors are designed so that each has a unique resonant frequency (e.g., fingerprint) that is spectrally far enough from each other to accommodate (and still distinguish) their frequency change when the strain builds up and their resonance frequency shifted. See FIG. 1, which schematically illustrates a plurality of wireless sensors f1-fn, each having individual operating frequencies that accommodate a Δf frequency shift margin, on a stress member. For this purpose, one or more architecture parameters of the unit design, namely the capacitive thin film thickness and/or the split width in the nested ring, can be intentionally varied in a sequence to create “n” dedicated spectral bins, each set by the initial resonance frequency “f1” to “fn” and each accommodating Δf frequency change margin/range.

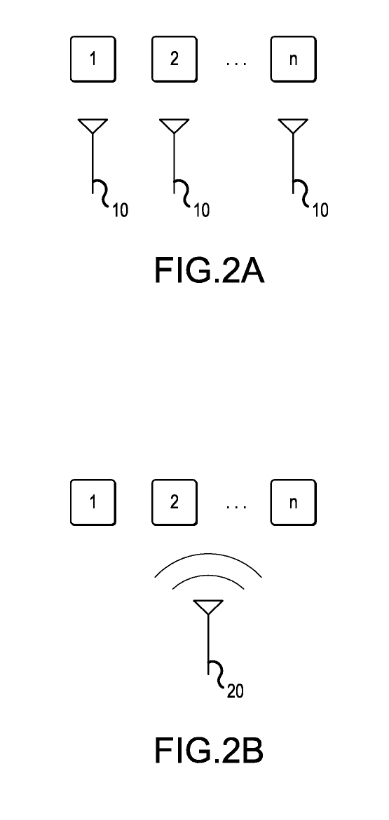

FIGS. 2A-2B schematically illustrates the wireless sensors f1-fn with different antenna schemes. Specifically, FIG. 2A schematically illustrates independent antennas each associated with one of the sensors. That is, each wireless sensor has its own antenna 10. The antennas 10 of the sensors f1-fn can be separately powered (i.e., externally excited) or together when the antennas 10 are connected in parallel.

FIG. 2B schematically illustrates a single antenna scheme that can send probing signals to and receive readouts from the f1-fn sensors. The multi-unit wireless strain sensors can be designed and fabricated directly on the stress member. To obtain each strain read-out signal, a single antenna 20 can be used to receive all strain data signals from all of the sensors concurrently, as illustrated in FIG. 2B.

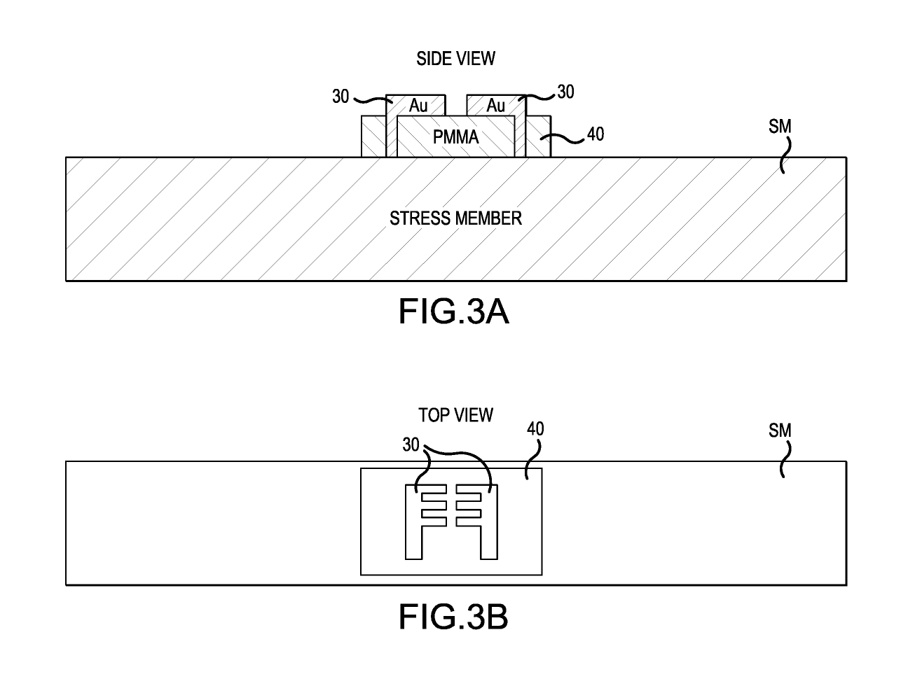

Referring to FIGS. 3-5, various wireless sensor architectures can be used to make the sensors, each of which is a strain dependent resonant passive sensor. Multi-finger split ring resonators (SRRs) are one example of the main architecture. Different implementations of these architectures are explained below.

FIGS. 3A-3B schematically illustrate one embodiment of the present device having f1-fn number of wireless sensors using a single metal layer structure (only one sensor shown for simplicity). Here the architecture is based on a single metal layer deposited structure. In this design, on any point, each side of the structured metal layer 30 has a multi-finger structure (SRR) connected to the metal stress member SM. In this example, the metal layer 30 is illustrated as gold as an example, and it is connected from the end of the nodes to the metal stress member SM. A solid dielectric film 40, such as Poly (methyl methacrylate) PMMA, can be used as a separation layer between the structured metal layer 30 that provides the multi-finger split ring configuration and the metal stress member SM. This separation can make the received and transmitted signal strength grow stronger.

Here the strain building on the stress member SM propagates through the solid dielectric layer 40 to enable the spacing between the fingers of the SRR structure to change. Changing the spacing length between the fingers shifts the resonance frequency, and the strain data signal can be read out from frequency response form each sensors concurrently since each sensor is designed to have a different resonance frequency by setting each sensor's finger number or the initial space between the fingers or both differently.

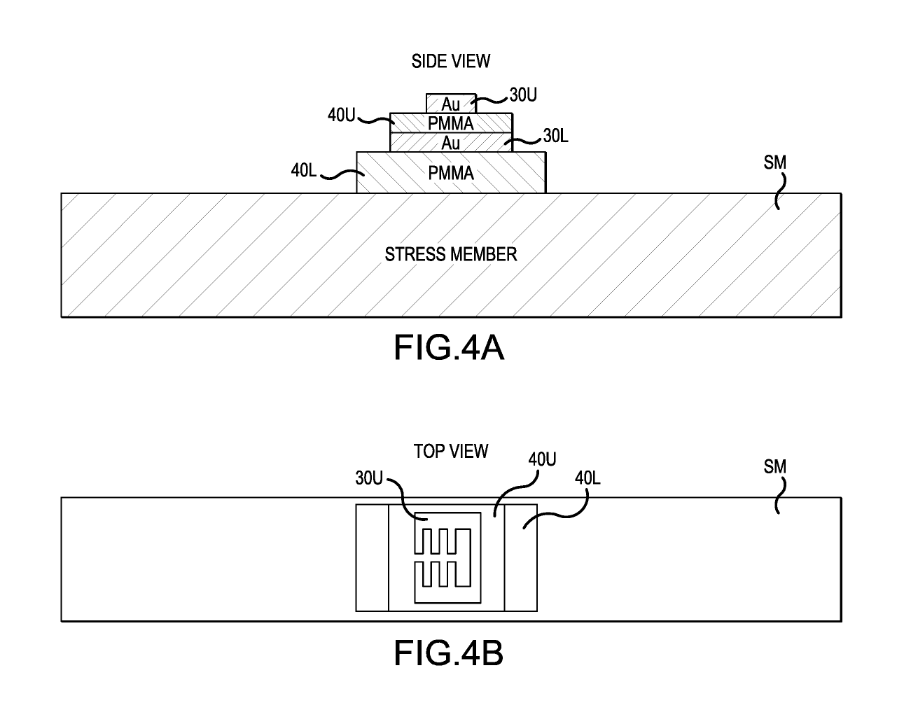

FIGS. 4A-4B schematically illustrate another embodiment similar to FIGS. 3A-3B, except that each wireless sensor uses a double metal layer structure with one patterned metal layer 30U and one unpatterned metal layer 30L. In this particular embodiment, two metal (e.g., gold illustrated) layers 30L, 30U are deposited over the solid dielectric layer 40L. Between these two metal layers, another solid dielectric film (separator), which can be made of PMMA or any other polymer-based material can be disposed as a dielectric layer 40U with a specific thickness. The first deposited metal (lower) layer 30L that is adjacent to the first solid dielectric layer 40L need not have any pattern. That is, the lower metal layer 30L can be a continuous metal film without any pattern. The lower metal layer 30L is used to establish the designed resonance frequency with the help of the thickness and permittivity value of the solid dielectric layer 40U.

Here, the strain on the stress member propagates through the sensor, changing the thickness of the upper solid dielectric layer 40U between the metal layers 30L, 30U, which causes the resonance frequency to shift. The change in the resonance frequency contains the strain data information.

Again, each sensor is designed to have different resonance frequencies by controlling each sensor's finger numbers and/or the initial thickness of the solid dielectric layer 40U between the metal layers 30L, 30U.

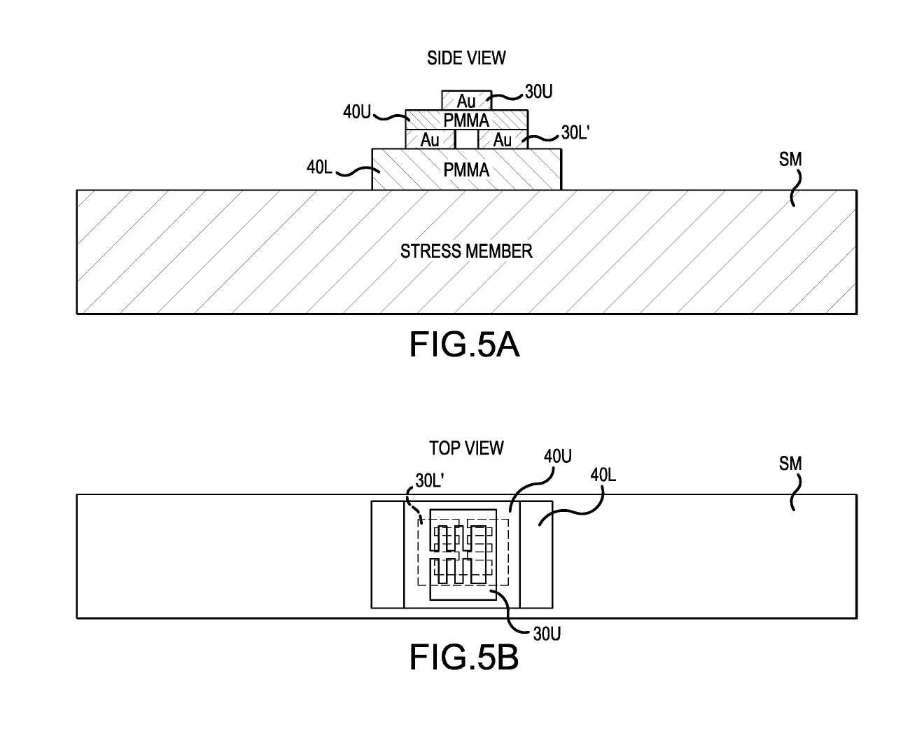

FIGS. 5A-5B schematically illustrate another embodiment similar to FIGS. 4A-4B, except that each sensor uses a double metal layer structure with two patterned metal layers 30U, 30L′. The embodiment of FIGS. 5A-5B is similar to the embodiment of FIGS. 4A-4B, except that the lower metal layer 30L′ is also patterned. The lower metal layer 30L′ can have the same size patterned structure as the upper metal layer 30U. The upper metal layer 30U can be stacked so that the spacing between fingers are 90° offset with the spacing between the fingers of the lower layer 30L′ as illustrated in FIG. 5. The working principle is the same as the embodiment of FIGS. 4A-4B.

The wireless sensors can be fabricated or printed directly on the stress member SM. The surface of the stress member SM, if not sufficiently smooth, can be polished to enable fabrication with conventional lithography equipment used for fabrication semiconductors.

It is important to first provide a film of solid dielectric material, such as PMMA or similar polymer-based materials on the stress member surface. The PMMA material is also bio compatible so that the present device can be used as an implant device. The dielectric coating also reduces the surface roughness for the subsequent standard microfabrication process, such as evaporating metal structures typically used in lithography and hard mask to pattern the metal layer(s).

To enhance adhering properties between the adjoining layers, an interface layer can be provided between the adjoining layers. Each of the interface layers can be composed of titanium for example. Each of the metal layers can be made of gold or gold containing metal to enhance conductivity. For example, a Ti film can be thermally evaporated on the lower dielectric layer 40, 40L and an Au film 30, 30L, 30L′ can be similarly thermally evaporated on the Ti film, in this order, using a conventional thermal evaporation system. Next, lithography can be performed on the Au layer 30 (for a single metal layer structure), to pattern the sensor structure thereon, which pattern can be a coil or ring, comb-shape, using conventional lithography and metal lift-off (i.e., etching) to form the patterned conductive layer 30, 30U, 30L′.

The present development can obtain useful loading information, which can be successfully correlated with the stress or, if used as an implant device, the bone tissue healing and time evolution, which is critical to the proper assessment of the healing phase and the implant monitoring. This may not be possible without simultaneous or substantially concurrent multiple strain readings. The present development is therefore advantageous over all implants with no multiple reading capability.

All modifications and equivalents attainable by one of ordinary skill in the art from the present disclosure within the scope and spirit of the present invention are to be included as further embodiments of the present invention. It is to be understood that the above description is intended to be illustrative, and not restrictive, and that the phraseology or terminology employed herein is for the purpose of description. The scope of the present accordingly is to be defined as set forth in the appended claims.

What is claimed is:

1. A wireless strain sensing device comprising:
a stress member; and
a plurality of wireless resonating strain sensors disposed at different areas on the stress member,
wherein each of the wireless resonating stain sensors comprises a strain sensing circuit that functions as a strain gauge and a resonator and composed of:
a first dielectric layer composed of dielectric material disposed over the stress member; and
a first conductive layer disposed over the first dielectric layer, and
wherein each of the wireless resonating strain sensors is configured to have a resonant frequency that is spectrally separated from each other to enable each of the resonant frequencies of the wireless resonating strain sensors to be distinguishable from each other even when the strain applied to the stress member causes shifting of the resonance frequency thereof.

2. The wireless strain sensing device according to claim 1, wherein the first conductive layer has a patterned layout.

3. The wireless strain sensing device according to claim 2, wherein:
the stress member is made of a conductive material;
part of the first conductive layer is in electrical contact with the stress member through the first dielectric layer.

4. The wireless strain sensing device according to claim 1, wherein each of the wireless resonating strain sensors further includes:
a second dielectric layer disposed over the first conductive layer; and
a second conductive layer disposed the over the second dielectric layer.

5. The wireless strain sensing device according to claim 4, wherein the second conductive layer has a patterned layout.

6. The wireless strain sensing device according to claim 5, wherein the first conductive layer also has a patterned layout.

7. The wireless strain sensing device according to claim 1, wherein the stress member is made of metal.

8. The wireless strain sensing device according to claim 1, wherein the stress member is a conductive implant hardware.

9. The wireless strain sensing device according to claim 1, wherein the stress member is flexible and conductive.
